(19) United States
(12) Patent Application Publication
(10) Pub. No.: US 2024/0394541 A1
(43) Pub. Date: Nov. 28, 2024

(54) CONFORMAL TRAINING OF MACHINE- LEARNING MODELS

(71) Applicant: DeepMind Technologies Limited, London (GB)

(72) Inventors: Ali Taylan CEMGIL, London (GB); Arnaud DOUCET, London (GB); Krishnamurthy DVIJOTHAM, London (GB); David STUTZ, London (GB)

(21) Appl. No.: 18/695,765

(22) PCT Filed: Oct. 5, 2022
(86) PCT No.: PCT/EP2022/077703

§ 371 Date: Mar. 26, 2024

Related U.S. Application Data
(60) Provisional application No. 63/252,521, filed on Oct. 5, 2021.

(51) Int. Cl. G06N 3/084; G16H 10/60

(52) U.S. Cl. CPC G06N 3/084; G16H 10/60

(57) ABSTRACT

Methods, computer systems, and apparatus, including computer programs encoded on computer storage media, for training a classification machine-learning model. The system obtains calibration training examples and prediction training examples, determines a threshold value based on the calibration training examples, generates data characterizing predicted confidence sets based on the threshold value and the prediction training examples, and update model parameters based at least on the predicted confidence sets.

### CROSS-REFERENCE TO RELATED APPLICATION

This application claims priority to U.S. Provisional Patent Application No. 63/252,521, filed on Oct. 5, 2021, the disclosure of which is hereby incorporated by reference in its entirety.

### BACKGROUND

This specification generally relates to training machine-learning models to generate a prediction for an input, and in particular, to training machine-learning models to generate a conformal classification defining a confidence set that includes the true prediction with a user-specified confidence level.

A machine-learning model is configured to process, according to parameters of the machine-learning model, input data to generate an output that defines predictions or decisions. For example, neural networks are machine-learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with the current values of a respective set of parameters

The parameters of a machine-learning model can be determined through a training process based on training data that includes one or more training examples. For example, neural networks can be trained by updating the network parameters, including, e.g., weights and bias coefficients of the network layers of the neural network.

### SUMMARY

Conformal prediction provides an uncertainty estimate and a formal guarantee for an output prediction from an underlying machine-learning model, such as a classification model. For example, a conformal predictor can predict, for outputs generated by the classification model, a confidence set that contains the true classification with a user-specified confidence level. The predictive efficiency of the conformal predictor can be measured by the size of the confidence set, with a larger size of the confidence set indicating a decreased predictive efficiency.

This specification describes methods, computer systems, and apparatus, including computer programs encoded on computer storage media, for training a classification machine-learning model to be used with a conformal predictor to predict confidence sets.

In general, the classification model is configured to process a model input to generate a classification output that indicates, for each particular classification in a set of classifications, a predicted probability for the model input for the particular classification.

The classification model can be a classifier neural network configured to perform any of a variety of classification tasks. As used in this specification, a classification task is any task that requires the model to generate an output that includes a respective score (e.g., the predicted probability) for each of a set of multiple categories. The respective scores can be used to select one or more of the categories as a “classification” for the model input using the respective scores.

One example of a classification task is image classification, where the input to the classification model is an image, e.g., the intensity values of the pixels of the image, the categories are object categories, and the task is to classify the image as depicting an object from one or more of the object categories. That is, the classification output for a given input image indicates a prediction of one or more object categories that are depicted in the input image. As used herein, an image may refer to a still image or a moving image (e.g. video). The input to the classification model (i.e. image data) may comprise pixels of the image or another representation of the image, such as may have been produced by an encoder, e.g. an encoder neural network. An image may comprise color or monochrome pixel value data. Such images may be captured from an image sensor such as a camera or LIDAR sensor.

Another example of a classification task is text classification, where the input to the classification model is text and the task is to classify the text as belonging to one of multiple categories. One example of such a task is a sentiment analysis task, where the categories each correspond to different possible sentiments of the task. Another example of such a task is a reading comprehension task, where the input text includes a context passage and a question and the categories each correspond to different segments from the context passage that might be an answer to the question. Other examples of text processing tasks that can be framed as classification tasks include an entailment task, a paraphrase task, a textual similarity task, a sentiment task, a sentence completion task, a grammaticality task, and so on.

Other examples of classification tasks include audio classification tasks, such as speech processing tasks, where the input to the classification model is audio data representing speech. Examples of speech processing tasks include language identification (where the categories are different possible languages for the speech), hotword identification (where the categories indicate whether one or more specific “hotwords” are spoken in the audio data), and so on. The audio data may be obtained from a sensor, e.g. an audio transducer or microphone, and may comprise a representation of a digitized audio waveform e.g. a speech waveform. Such a representation may comprise samples representing digitized amplitude values of the waveform or, e.g., a time-frequency domain representation of the waveform such as an STFT (Short-Term Fourier Transform) or an MFCC (Mel-Frequency Cepstral Coefficient) representation. The output of the (audio) classification model may include predictions that audio signals indicative of different categories of audio signals are present in the input audio data (e.g. signals corresponding to an alarm sounding, a person speaking, a machine or vehicle being operated, etc.), in which case the output may be respective category scores indicative of a likelihood that the different audio signals are present in the audio data or a segment of the audio data. The classification task may include a speech or sound recognition task (e.g. the category scores may be indicative of a likelihood of different respective words being present in the audio data), a phone or speaker classification task (e.g. the category scores may each be indicative of a likelihood that different respective speakers were speaking in the audio data).

In general, the input data to the classification model may comprise a waveform (e.g. time series) of any signal, for example, a signal from a sensor, e.g. a sensor sensing a physical characteristic of an object in the real world (e.g. image data for the object). In some implementations, the sensor data may include, for example: data characterizing a state of a robot or vehicle, such as pose data and/or position/velocity/acceleration data: or data characterizing a state of an industrial plant, factory or data center such as sensed electronic signals such as sensed current and/or temperature signals. The classification task may include an event or state tagging task, in which case the output of the classification model may include respective category scores indicative of whether different events or different states (e.g. of the environment or equipment in the environment) are represented in the input data or segment of the input data. In some examples, such events or states may include operating states of a device or machine, and/or transitions between the different states, e.g. transitions between an expected or functioning state to an unexpected or non-functioning state of a device or machine. The classification model output may include respective category scores for different categories indicative of physical characteristics (e.g. size, position, velocity) or states of an object or the environment.

The conformal predictor can be used to assess the uncertainty of the classification predictions outputted by the classification machine-learning model. For any appropriate classification model applied to any classification task, the predictor is configured to process an input including the classification output from the classification model to generate data that identifies, for the model input to the classification model, a confidence set that includes one or more output classifications selected from the set of classifications such that a probability of a true classification of the model input being included in the confidence set is greater than or equal to a confidence level, e.g., a user-defined confidence level. That is, the classification accuracy of the confidence set outputted by the conformal predictor (in the sense that the confidence set includes the true classification) is guaranteed to follow the confidence level, which can be user-defined according to a specific application.

The above property of the conformal predictor is important for achieving reliability for the conformal classification machine-learning model, especially in high-stake applications such as medical diagnosis, self-driving vehicles, and robotics. For example, in computer-aided cancer detection, it may be more important and useful to capture a set of potential diagnoses with high confidence (e.g., a 99% confidence level) than to provide the most likely single diagnosis without providing a guarantee for reliability.

In one example, the classification model and the conformal predictor are used for a medical screening and diagnosis task, in which the input to the model is medical data characterizing a user's health and the model output is a prediction over a plurality of categories that each represents a different diagnosis. The model input can include medical images, e.g., one or more medical scan images, X-ray, CT, MRI, or ultrasound images of the subject. Alternatively or additionally, the model input can include microscopic histology images from biopsied tissues. Alternatively or additionally, the model input can include physiological parameters of the subject, such as the BMI, blood pressure, heart rate, diabetic milieu, serum levels of various hormones, and genetic markers of the subject, and symptom information for the subject, such as the location and severity of discomfort in the body. Alternatively or additionally, the model input can optionally include other information characterizing the subject. The classification model can be configured to process the model input to predict probability scores for multiple diagnostic categories. For example, when the medical diagnosis task is cancer, e.g., breast cancer, screen, and detection, the diagnostic categories can include different diagnoses for breast cancer screening and detection, including, for example, “Normal”, “Adenosis”, “Fibroadenoma”, “Ductal Carcinoma”, “Tubular Carcinoma”, “Lobular Carcinoma”, etc. Since missing an early diagnosis of a malignant breast tumor such as an invasive ductal carcinoma may cause a significantly worsened prognosis for the patent, it may be important to include a set of possible diagnoses in the prediction with a guaranteed confidence level to guide follow-up tests and interventions. For example, on the one hand, the classification output may indicate that the most likely diagnosis is a benign breast tumor “Fibroadenoma” (e.g., with a 0.6 predicted probability). On the other hand, the confidence set predicted by the conformal predictor with a 95% confidence level may include {“Fibroadenoma”, “Ductal Carcinoma”, “Tubular Carcinoma”}. Thus, the predicted confidence set provides more information than the classification output alone, since the malignant tumors included in the predicted confidence set should cause concern and follow-up while simply viewing the highest-scoring category will not indicate any cause for concern.

In another example, the classification model and the conformal predictor are used on-board an autonomous vehicle. In this example, the input to the model can be sensor data captured by the sensor(s) of the vehicle, e.g., image data, Lidar data, radar data, or some combination and the classification model output is a respective score for each of a plurality of object categories that represent different types of objects, e.g., vehicles, pedestrians, traffic signs, cyclists, and so on. Since missing the detection of a pedestrian or cyclist in the scene can cause catastrophic mistakes in the vehicle's maneuver, it may be important to include a set of possible classifications of an object in the prediction with a guaranteed confidence level to guide the vehicle maneuver. For example, on the one hand, the classification output may indicate that the most likely classification is a traffic sign. On the other hand, the confidence set predicted by the conformal predictor with a 95% confidence level may include a pedestrian and/or a cyclist. Thus, the predicted confidence set provides more information than the classification output alone, since the pedestrian and/or cyclist included in the predicted confidence set should signal caution in the vehicle maneuver, while simply viewing the highest-scoring category will not indicate any cause for concern.

In one innovative aspect, this specification describes a method for training the conformal classification machine-learning model. The method is implemented by a system including one or more computers.

The system obtains a set of calibration training examples and uses the calibration training examples to determine a threshold value. Each calibration training example includes a respective training model input and a respective classification label for the respective training model input. For each respective calibration training example in the set of calibration training examples, the system processes the respective training model input of the respective calibration training example using the classification model, according to current values of the model parameters, to generate a respective classification output for the respective training model input. The system determines the threshold value based at least on the classification outputs generated for the calibration training examples, the classification labels in the calibration training examples, and a confidence level.

The system further obtains a set of prediction training examples. Each prediction training example includes a respective training model input and a respective classification label for the respective training model input. For each respective prediction training example in the set of prediction training examples, the system processes the respective model input of the respective prediction training example using the classification model, according to the current values of the model parameters, to generate a respective classification output, and processes an input including the respective classification output and the threshold value with a smooth prediction function to generate a respective prediction output. The smooth prediction function is differentiable with respect to the input to the smooth prediction function (e.g. one or more derivatives of the smooth prediction function with respect to the input may be a continuous function).

The system determines a gradient with respect to the model parameters of a training loss. The training loss includes an inefficiency loss that at least measures, for each respective prediction training example, a value indicating a size of the respective predicted confidence set characterized by the respective prediction output. The system updates the current values of the parameters of the model parameters using the gradient.

In some implementations, the respective prediction output includes a confidence score for each classification in the set of classifications. The respective confidence score characterizes a respective probability of the respective classification being included in the predicted confidence set.

In some implementations, the classification model includes a neural network with any appropriate architecture, such as the multi-layer perceptrons or the ResNets.

In some implementations, the inefficiency loss measures a weighted size that is computed based on the sizes of the respective predicted confidence sets scaled by weight coefficients for the set of classifications.

In some implementations, the training loss further includes a classification loss that measures a classification error of the output classifications included in the predicted confidence set characterized by the respective prediction output.

The classification loss can include a first term that measures, for each respective prediction training example, an error of not including the true classification in the predicted confidence set characterized by the prediction output.

In implementations, the model input may comprise sensor data (e.g. image, video and/or audio data), and the classifications correspond to object categories; and/or the model input may comprise medical data, such as physiological measurements, of a patient and the classifications each correspond to a different diagnosis for the patient; and/or the model input may comprise observations of the environment and the classifications each correspond to a different state of the environment.

The classification loss can also include a second term that measures, for each respective prediction training example, an error of including a specified classification that is not the true classification in the respective output confidence set. The specified classification can be determined, based on priori information (i.e. prior information or information known “a priori”, e.g. information that is separate from or in addition to the information contained in the model input for determining the classification output), as a classification contradictory to the true classification. For example, in the case of medical diagnosis classifications, it may be desirable to avoid a high-risk condition, such as “Ductal Carcinoma” in the confidence set when the ground truth label is “Normal”. This is motivated by avoiding unwanted anxiety or tests for the patient. Thus, the second term of the classification loss can be used to penalize certain high-risk classifications when the true label indicates a low-risk classification.

In some implementations, the prediction function includes a smoothed implementation of a threshold function applied to a difference between a first value computed based on the classification output and a second value computed based on the threshold value. In one example, for each respective prediction training example in the set of prediction training examples, the first value can be computed as the predicted probability indicated by the respective classification output for a particular classification. In another example, for each respective prediction training example in the set of prediction training examples, the first value can be computed as a sum of a plurality of predicted probabilities indicated by the respective classification output for a plurality of classifications from the set of classifications. The smoothed threshold function can be a sigmoid function.

In some implementations, in determining the threshold value, the system processes the classification outputs from the classification model, the classification labels in the calibration training examples, and the confidence value with a smooth calibration function that outputs the threshold value. The smooth calibration function is differentiable with respect to the classification outputs. The calibration function can be configured to: compute, for each respective calibration training example in the set of calibration training examples, a respective conformity score based on the respective classification output and the respective classification label, and perform a smoothed implementation of a quantile operation on the conformity scores and the confidence level.

In some implementations, the system further obtains a batch of training examples, and randomly samples the batch of training examples to generate the set of calibration training examples and the set of prediction training examples. The system can also obtain a plurality of batches of training examples, and repeatedly perform training of the classification machine-learning model on each of the batches of training examples.

This specification also describes a method for performing conformal classification. The method is implemented by a system including one or more computers. The system obtains a model input, obtains the classification model that has been trained using any of the methods described above, and processes the model input with the classification model to generate a classification output that indicates, for each particular classification in a set of classifications, a predicted probability for the model input for the particular classification. The system further obtains a threshold value that has been predicted by a non-smooth calibration function based on a calibration data set and according to a confidence value, and processes the model output and the threshold value to generate a confidence set that can include one or more output classifications selected from the set of classifications. (The output confidence set can be empty in some cases if none of the classifications satisfy the confidence value condition.)

In implementations, the method may further comprise, (automatically) performing, based at least in part on the confidence set, one or more of: controlling (i.e. providing instructions to) a robot or a vehicle (e.g. an autonomous or semi-autonomous land or air or sea vehicle), wherein the model input comprises sensor data (e.g. image, video and/or audio data) obtained by one or more sensors (e.g. sensors of the robot or vehicle) and the classifications correspond to object categories (e.g. categories of objects that the robot or vehicle may interact with or manipulate, or which are present in the environment surrounding the robot or vehicle): providing one or more medical diagnoses, wherein the model input comprises medical data (e.g. medical images), such as physiological measurements, of a patient and the classifications each correspond to a different diagnosis for the patient; and controlling an agent to perform a task in an environment, wherein the model input comprise observations of the environment (e.g. sensor data obtained from sensors of the environment) and the classifications each correspond to a different state of the environment.

In some implementations, the robot (e.g. a static or moving machine) interacts with a real-world environment to accomplish a specific task, e.g., to locate an object of interest in the environment or to move an object of interest to a specified location in the environment or to navigate to a specified destination in the environment.

In some implementations, the agent may, based at least in part on the confidence set, perform an action in a real-world (or a simulated) environment. The environment may include items of equipment, for example in a facility such as: a data center, server farm, grid mains power or water distribution system, or in a manufacturing plant or service facility. The observations may then relate to the operation of the plant or facility. For example additionally or alternatively they may include observations of power or water usage by equipment, observations of power generation or distribution control, or observations of usage of a resource or of waste. The agent may perform actions in the environment to increase efficiency, for example by reducing resource usage, and/or reducing the environmental impact of operations in the environment, for example by reducing waste, and/or for safety reasons (e.g. to prevent harm to equipment and/or users). The actions may include actions controlling or imposing operating conditions on items of equipment of the plant/facility, and/or actions that result in changes to settings in the operation of the plant/facility e.g. to adjust or turn on/off components of the plant/facility.

This specification also describes a system including one or more computers and one or more storage devices storing instructions that when executed by the one or more computers cause the one or more computers to perform the methods described above.

This specification also describes one or more computer storage media storing instructions that when executed by one or more computers, cause the one or more computers to perform the methods described above.

In situations in which the systems discussed here collect information about users, or may make use of such information, the users may be provided with an opportunity to control whether the programs or features collect user information. In addition, certain information may be treated in one or more ways before it is stored or used in an effort to remove personally identifiable information therefrom. Thus, the user may have control over how information is collected about the user and used by systems described herein.

The subject matter described in this specification can be implemented in particular embodiments so as to realize one or more of the following advantages.

Although modern deep-learning based classifiers show very high accuracy on test data, they do not provide guarantees for safe deployment, especially in high-stake machine-learning applications such as medical diagnosis and automatic driving. For conventional machine-learning classification models, predictions are obtained without a reliable uncertainty estimate or a formal guarantee. Conformal prediction addresses these issues by using the classifier's predictions, e.g., its probability estimates, to predict confidence sets containing the true class with a user-specified probability.

Typically, conformal prediction is used as a separate processing step after the machine model is trained. That is, the machine-learning model is not trained with the objective of predicting the optimal confidence set. For example, the machine-learning classification model is typically trained to minimize a cross-entropy loss without taking into account of factors such as reducing the expected size of the confidence set outputted by the conformal prediction function. After the classification model has been trained, its parameters are fixed as the conformal predictor is used to compute the confidence set, leaving the conformal predictor little to no control over the predicted confidence sets, such as the size (inefficiency) or composition (i.e., the included classes) of the confidence set.

In order to overcome the limitations of conventional conformal prediction methods, this specification provides techniques for training the classification model with the conformal predictor end-to-end. By developing a differentiable model for conformal prediction and training the classification model with the objective computed based on the predicted confidence set, the described techniques allow the optimization of specific objectives defined on the predicted confidence sets without losing the guarantees. For example, the provided techniques can be used to reduce inefficiency, i.e., decrease the size of the predicted confidence set, and thus generate conformal predictions that provide better guidance in decision-making based on predicted classifications. The reduction in inefficiency of the outputted confidence set can benefit many practical applications. For example, in medical diagnosis, smaller confidence sets are important for avoiding confusion and anxiety among doctors and patients, ultimately leading to better diagnoses and improved decision making for medical interventions.

Further, the described techniques allow shaping the confidence sets according to the application scenario, by designing the inefficiency loss and/or the classification loss used for training. This allows the system to better exploit priori information, such as the importance of a specific classification, and/or relationships between different classifications. For example, in the context of medical diagnosis, the provided techniques can be used to particularly reduce inefficiency, i.e., uncertainty, on low-risk diseases. This may guide the doctors to focus more on high-risk conditions. Alternatively or additionally, the described techniques can be used to enforce constraints on the composition of the confidence set so that the confidence set is less likely to include two conditions that are frequently confused by doctors, or to include both a high-risk condition and a low-risk condition. These flexibilities in shaping the optimization objectives of the confidence set may be advantageous in specific scenarios for further improving the diagnoses and decision-making.

### BRIEF DESCRIPTION OF THE DRAWINGS

FIG. 1 shows an example conformal training system for training a classification model.

FIG. 2 shows an example of performing conformal training of a classification machine-learning model.

FIG. 3 shows an example conformal classification system.

FIG. 4 is a flow diagram illustrating an example process for training a classification model.

FIG. 5 is a flow diagram illustrating an example process for performing conformal classification.

FIG. 6 shows the performance comparison between classification models trained using conformal training and classification models without conformal training in predicting the confidence set.

Like reference numbers and designations in the various drawings indicate like elements.

### DETAILED DESCRIPTION

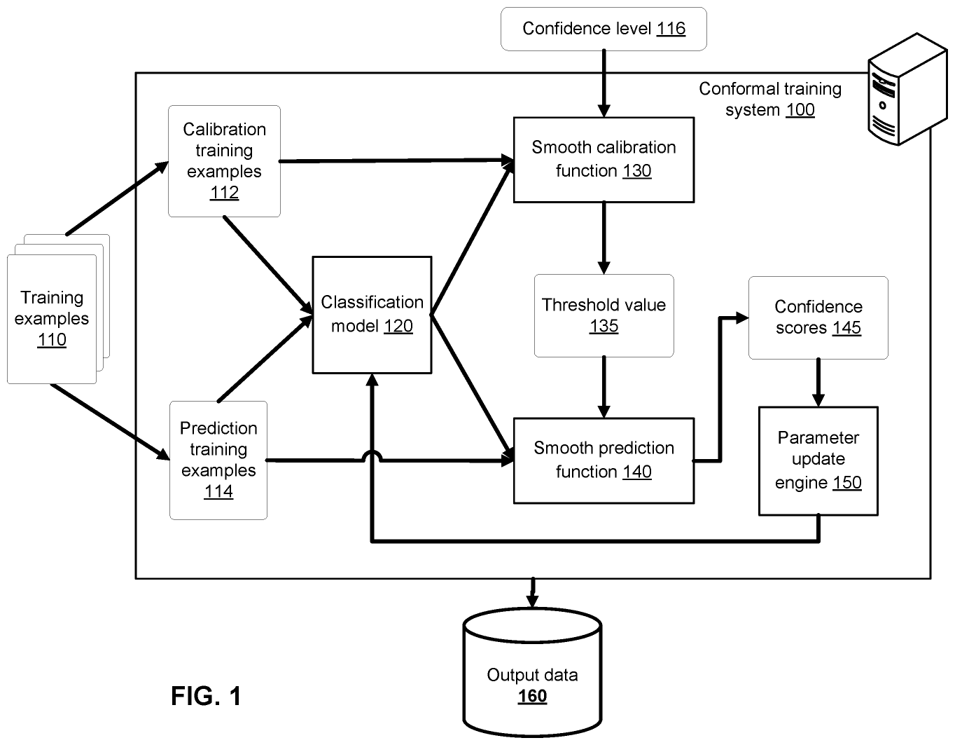

FIG. 1 shows an example of a conformal training system 100 for training a machine-learning model, e.g., a classification machine-learning model 120. The system 100 is an example of a system implemented as computer programs on one or more computers in one or more locations, in which the systems, components, and techniques described below can be implemented.

In general, the classification machine-learning model 120 is configured to perform a classification task, i.e., to process input data to generate output data that specifies predicted classification information of the input data. The input data can specify any type of data to be analyzed, e.g., an image, a text, an audio signal, a sensor measurement, event log, and so on. The output data can specify classification information for the input data, e.g., one or more object categories of objects being depicted by an input image, one or more text categories of an input text, one or more status categories of a system, one or more health condition categories of a person, and so on.

The classification model 120 can be any appropriate machine-learning model for performing a classification task. In some implementations, the classification model 120 can be a classifier neural network with any appropriate architecture configured to perform any of a variety of classification tasks. For example, the classifier neural network can be used to process an input to generate an output that includes a respective score (e.g., a predicted probability) for each of a set of multiple classifications. The respective predicted probabilities can be used to select one or more of the categories as a “classification” for the model input.

The goal of the conformal training system 100 is to optimize the parameters (e.g., network weight and bias coefficients) of the classification model 120 for one or more optimization measures of a confidence set predicted based on a confidence level 116. This process may be referred to as conformal training of the classification model 120. The predicted confidence set includes, for a model input of the classification model 120, one or more output classifications selected from the set of multiple classifications such that a probability of a true classification of the model input being included in the confidence set is greater than or equal to the confidence level 116. That is, the prediction accuracy of the confidence set (in the sense that the confidence set includes the true classification) is guaranteed to satisfy the confidence level, which can be user-defined according to a specific application. As will be discussed in the following, the optimization measures of the confidence set can be configured to aim for minimizing the inefficiency, i.e., the size of the confidence set while the coverage of the true classification is guaranteed with confidence level 116.

The training process performed by the system 100 is different from training a classification model in the conventional conformal classification process. In conventional conformal classification, the classification model is pre-trained on training data without considering properties of the predicted confidence set. For example, the classification model can be trained on a cross-entropy loss only. After the classification model has been trained, a conformal calibration process is applied to the classification model (with its model parameters being fixed to the trained values) on a calibration dataset to determine a threshold value t for conformity scores computed based on the predicted classification probabilities. The conformity score can be understood as a measure quantifying the conformity or non-conformity (or “strangeness”) of a data sample in a given data set. The threshold value t can be determined through analyzing the data distribution of the classification model output on the calibration data set with respect to the user-specified confidence level. The threshold value t then can be used to compare with the conformity scores computed for a classification model output to determine the confidence set.

By contrast, in the conformal training process performed by the system 100, the classification model 120 is trained end-to-end with the conformal calibration (to determine the threshold value t 135) and the conformal prediction (to predict the confidence set). As a result, the training process optimizes the properties of the predicted confidence set directly. This process offers several advantages over conventional conformal classification, as will be discussed in the following.

The system 100 obtains a plurality of training examples 110. Each training example includes a training model input, e.g., xᵢ, and a respective ground-truth classification label, e.g., yᵢ, which is the ground truth classification of xᵢ. In some implementations, the system 100 can perform conformal training of the classification model 120 using stochastic gradient descent on mini-batches of the set of training examples 110. For each batch of training examples, the system 100 can split the batch (e.g., by randomly sampling the batch) into a set of calibration training examples 112 and a set of prediction training examples 114. In general, the calibration training examples 112 and the set of prediction training examples 114 are disjoint sets. As described in the following, the system 100 may use the calibration training examples 112 in a calibration process to determine the threshold value t for conformity scores. The system 100 may use the prediction training examples 114, along with the threshold value t, in a prediction process to predict data characterizing the predicted confidence set for each prediction training example 114. The predicted confidence sets may be used to compute a training loss for updating the model parameters of the classification model 120.

In the calibration process, for each calibration training example 112, e.g., (xᵢ,yᵢ), the system 100 processes the respective training model input xᵢ using the classification model 120, e.g., π_{θ}, according to current values of the model parameters θ, to generate a respective classification output π_{θ}(xᵢ) for the respective training model input xᵢ. For example, if the set of multiple classifications includes K classifications [K], the classification output π_{θ}(xᵢ) can include K predicted probabilities {π_{θ,k}(x)} for k∈[K].

For each calibration training example (xᵢ, yᵢ), the system 100 computes the conformity score E_{θ}(xᵢ, yᵢ) of the model output π_{θ}(x) based on the ground-truth label yᵢ.

In some implementations, following the threshold conformal predictor (THR) formulation, the conformity score for a classification model output is defined as

$${E}_{θ} ( x , k ) := {π}_{θ , k} ( x ) . \qquad ( 1 )$$

That is, the conformity score for the particular classification k may be defined as the predicted probability π_{θ,k}(x) for the classification k in the classification model output π_{θ,k}(x). In conventional conformal classification using the THR formulation, the confidence set is determined with respect to the threshold value τ as

$${C}_{θ} ( x ; τ ) := \{ k : {E}_{θ} ( x , k ) := {π}_{θ , k} ( x ) \geq τ \} . \qquad ( 2 )$$

That is, if the predicted probability for a particular classification is greater or equal to the threshold value, the particular classification is selected to be included in the confidence set C_{θ}(x;τ). Details of the THR formulation can be found in Sadinle, et al., “Least ambiguous set-valued classifiers with bounded error levels,” Journal of the American Statistical Association (JASA), 114 (525): 223-234.

Also following the THR formulation, the system 100 may compute, for each calibration training example (xᵢ,yᵢ), the conformity scores E_{θ}(xᵢ,yᵢ) of the model output π_{θ}(xᵢ) with respect to the ground-truth label yᵢ as the predicted probability in the classification output of the ground-truth classification of the model input, that is,

$${E}_{θ} ( {x}_{i} ) := {π}_{θ , {y}_{i}} ( {x}_{i} ) . \qquad ( 3 )$$

In practice, the conformity scores may also be defined as the logits (THRL) or log-probabilities (THRLP) instead of probabilities.

In conventional conformal prediction using the THR formulation, for a confidence level 1−α (where α is the significance level that limits the error rate of the predicted confidence set), the threshold value τ is computed as

α(1+1/|I_{cal}|)-quantile of the conformity scores E_{θ}(xᵢ,yᵢ)=π_{θ,y}_{ᵢ}(xᵢ), (4)
where |I_{cal}| is the size of the calibration training example set 112. The quantile operation in the conventional THR causes the threshold value t to be non-differentiable with respect to the classification outputs π_{θ}(xᵢ).

In order to enable training the classification model 120 and the conformal predictor end-to-end, the system 100 uses the smooth calibration function 130 to perform a smoothed i.e., differentiable implementation of the quantile operation on the conformity scores, so that the computed threshold value t is differentiable with respect to the classification outputs for the calibration examples {(π_{θ}(xᵢ), yᵢ}_{i∈I}_{_{cal}} and thus differentiable with respect to the model parameters θ. This can be accomplished using any appropriate smooth sorting techniques. One example of the sorting technique is described in Blondel, et al., “Fast differentiable sorting and ranking,” Proc. of the International Conference on Machine Learning (ICML), 2020. Another example of the sorting technique is described in Cuturi, et al., “Differentiable ranking and sorting using optimal transport,” Advances in Neural Information Processing Systems (NeurIPS), 2019. These smooth sorting implementations often include a “dispersion” hyper-parameter e such that smooth sorting approximates “hard” sorting for ε→0.

In some implementations, following the adaptive prediction sets (APS) formulation, the conformity score for a classification model output is defined as

$${E}_{θ} ( x , k ) := {π}_{θ , y}^{( 1 )} ( x ) + … + {π}_{θ , y}^{( k - 1 )} ( x ) + U {π}_{θ , y}^{( k )} ( x ) , \qquad ( 5 )$$

where π_{θ,y}_{⁽¹⁾}(x)≥ . . . ≥π_{θ,y}_{^{(K)}}(x) and U is a uniform random variable in [0,1] to break ties. That is, the conformity score for a particular classification k is defined as a sum of ranked (from large to small) predicted probabilities π_{θ,k}(x) with the smallest predicted probability being scaled by a uniform random variable.

In conventional conformal classification using the APS formulation, the confidence set is determined with respect to the threshold value τ as

$${C}_{θ} ( x ; τ ) := \{ k : {E}_{θ} ( x , k ) \leq τ \} . \qquad ( 6 )$$

That is, based on Eq. 5 and Eq. 6, after the predicted probabilities for the set of classifications are ranked from large to small, a particular classification k is selected into the confidence set C_{θ}(x;τ) if the particular classification k belongs to the subset of top-ranked probabilities whose sum is below or equal to the threshold value τ. Details of the APS formulation can be found in Romano, et al., “Classification with valid and adaptive coverage,” Advances in Neural Information Processing Systems (NeurIPS), 2020.

In the conventional conformal prediction using the APS formulation, for a confidence level 1−α, the threshold value τ is computed as

$$( 1 - α ) ( 1 + 1 / \vert {I}_{c a 1} \vert ) - quantile of the conformity scores {E}_{θ} ( {x}_{i} , {y}_{i} ) . \qquad ( 7 )$$

Similar to the smoothed implementation of the THR formulation for computing the threshold value τ, the system 100 uses the smooth calibration function 130 to perform the smoothed implementation of the quantile operation in the APS formulation for computing threshold value τ, so that the computed threshold value τ is differentiable with respect to the classification output for the calibration examples {(π_{θ}(xᵢ), yᵢ}_{i∈I}_{_{cal}} and thus the model parameters θ.

Although the THR and the APS formulations are described as examples above, any other suitable formulations for conformal calibration can be used as long as the formulation can be adopted or converted into a differentiable form to be implemented by the smooth calibration function 130.

After the threshold value (t) 135 has been determined by the smooth calibration function 130 based on the calibration training examples 112, the system 100 uses the smooth prediction function 140 to generate data characterizing the predicted confidence sets. In particular, the system can use the smooth prediction function 140 to generate confidence scores 145 based on the prediction training examples 114 and the threshold value 135. The confidence scores 145 can be understood as characterizing the probabilities of the respective classifications being included in the confidence set.

For each prediction training example 114, e.g., (xᵢ,yᵢ), the system 100 processes the respective training model input xᵢ using the classification model 120, e.g., π_{θ}, according to current values of the model parameters θ, to generate a respective classification output π_{θ}(xᵢ) for the respective training model input xᵢ. Then the system 100 generates the respective conformity score for each classification E_{θ}(xᵢ,k) using the formulation chosen in the smooth calibration process, and uses the smooth prediction function 140 to generate confidence scores 145.

In conventional conformal prediction, the confidence set is determined by performing thresholding operations on the conformity scores over the threshold value 135. The thresholding operations, as well as the typically discrete numbers representing the classifications selected into the confidence set, are not differentiable.

In order to enable training the classification model 120 and the conformal prediction end-to-end, the system 100 performs a smoothed, i.e., differentiable implementation of the thresholding operation. That is, the system 100 applies a smooth threshold function to a difference between a first value computed based on the conformity scores E_{θ}(x,k) and a second value computed based on the threshold value τ. Any appropriate smooth thresholding function can be used. In one particular example, a sigmoid function σ can be applied to the difference to compute the confidence scores 145 as:

$${C}_{θ , k} ( x ; τ ) := σ ( ( {E}_{θ} ( x , k ) - τ ) / T ) , \qquad ( 8 )$$

where T is a temperature hyper-parameter.

The confidence scores C_{θ,k}(x;τ)∈[0,1] represents a soft assignment of classification k to the confidence set, i.e., can be interpreted as the probability of k being included in the confidence set. For T→0, the “hard” confidence set will be recovered, i.e., C_{θ,k}(x;τ)=1 for k∈C_{θ}(x;τ) and 0 otherwise.

The above confidence scores C_{θ,k}(x;τ) computed using a smooth thresholding function are differentiable with respect to the conformity scores E_{θ}(x,k) and the threshold value τ. Thus, the confidence scores C_{θ,k}(x;τ) are differentiable with respect to the model parameters θ as long as the conformity scores E(x,k) are defined as differentiable functions of the classification outputs π_{θ}(xᵢ), since the threshold value τ 135 outputted by the smooth calibration function 130 is differentiable with respect to the classification output.

After the confidence scores 145 have been computed based on the prediction training examples 114 and the threshold values, the system uses the parameter update engine 150 to update the model parameters of the classification model 120. For example, the parameter update engine 150 can determine a gradient with respect to the model parameters of a training loss computed on the confidence scores C_{θ,k}(x;τ), and update the current values of the model parameters using any appropriate backpropagation approaches using the gradient.

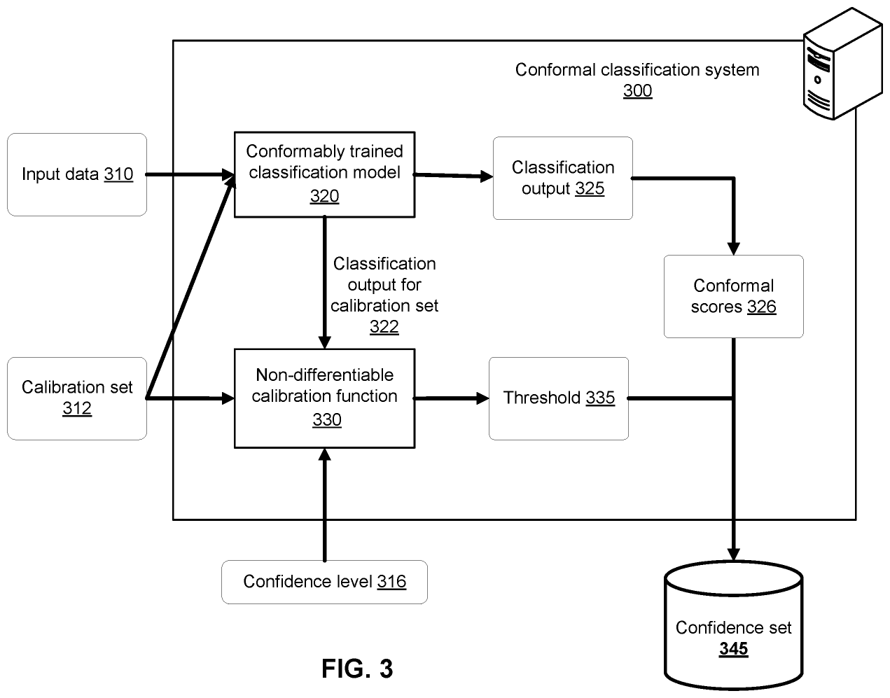

As will be described with reference to FIG. 3, after the classification model 120 has been conformally trained, i.e., trained on the training loss computed based on the confidence scores, the trained classification model 120 can be used with conventional conformal calibration and conformal prediction to predict the confidence set based on a user-defined confidence level. That is, a conventional non-smooth conformal calibration process (of any appropriate formulation) can be performed on a calibration dataset to determine the threshold value τ based on the user-defined confidence level, so that coverage can be guaranteed with the user-defined confidence level. In some implementations, the threshold value 135 can be computed based on the same calibration data set that has been used for training the classification model 120. However, instead of using the “soft” sorting in the smooth calibration function for training the classification model 120, the threshold value 135 used for predicting the confidence set is obtained using a “hard” sorting to guarantee coverage.

In general, the training loss includes an inefficiency loss [symbol] _{ineff} that at least measures, for each respective prediction training example, a value indicating a size of the respective predicted confidence set in the respective prediction output generated for the respective prediction training example. That is, the inefficiency loss [symbol] _{ineff} can be defined as:

$${L}_{ineff} = Ω ( {C}_{θ} ( x ; τ ) ) , \qquad ( 9 )$$

where Ω is a “smooth” size function of the expected inefficiency of the confidence set, e.g., [symbol] [|C_{θ}(X;τ)|]. Since the confidence score C_{π,k}(x;τ) can be understood as a soft assignment of class k to the confidence set C_{θ}(x;τ), in some implementations, the inefficiency loss [symbol] _{ineff} can be computed based on a sum of the confidence scores for the set of classifications, e.g., as:

$${L}_{ineff} = max ( 0 , {\sum }_{k = 1}^{K} {C}_{θ , k} ( x ; τ ) - κ ) , \qquad ( 10 )$$

where by default κ=1 in order to not penalize singletons. In general, κ∈{0,1} can be treated as a hyperparameter.

In some implementations, the training loss further includes a classification loss [symbol] _{class} that measures a classification error of the output classifications included in the predicted confidence set characterized by the respective prediction output. The total training loss can be computed as

$$ℒ = {ℒ}_{c l a s s} + {λℒ}_{ineff} , \qquad ( 11 )$$

where λ is a hyperparameter.

In some implementations, the classification loss [symbol] _{class} can be directly computed on the confidence sets C_{θ} to explicitly enforce coverage, i.e., make sure the ground-truth label Y is included in C_{θ}(X; τ), and optionally penalize other classes k not to be included in C_{θ}. To this end, in one implementation, the classification loss [symbol] _{class} can be defined as

$${ℒ}_{c l a s s} ( {C}_{θ} ( x ; τ ) , y ) := \text{} {\sum }_{k = 1}^{K} {L}_{y , k} [ \underbrace{( 1 - {C}_{θ , k} ( x ; τ ) ) \cdot δ [ y = k ]}_{enforce y to be in C} + \underbrace{{C}_{θ , k} ( x ; τ ) \cdot δ [ y \neq k ]}_{penalize class k \neq y not to be in C} ] . \qquad ( 12 )$$

where L_{y,k}[·] is a loss matrix.

As described above, the confidence scores C_{θ,k}(x;τ)∈[0,1] such that 1−C_{θ}(x;τ) can be understood as the likelihood of k not being in C_{θ}(x;τ). The first term of [symbol] _{class} is used to encourage coverage, while the second term can be used to avoid predicting other classes. This is governed by the loss matrix L_{y,k}[·]. For L=I_{K}, i.e., the identity matrix with K rows and columns, the loss matrix simply enforces coverage. On the other hand, setting any L_{y,k}>0 for y≠k penalizes the model from including class k in confidence sets with ground truth y. Thus, defining L allows to define complex objectives according to specific applications.

In skin condition classification, for example, predicting sets of classes, e.g., the top-k conditions, is already a common strategy for handling uncertainty. In these cases, in addition to the coverage guarantees, characteristics of the confidence sets can be also important. The constraints in terms of the predicted confidence sets cannot be readily handled by conventional conformal classification. The conformal training performed by system 100, on the other hand, allows defining complex objectives to enforce constraints on the predicted confidence set.

For example, the system can reduce inefficiency, i.e., uncertainty, on “low-risk” diseases at the expense of higher uncertainty on “high-risk” conditions. This can be thought of as re-allocating time spent by a doctor towards high-risk cases. Using conformal training, the system can manipulate group- or class-conditional inefficiency using a weighted size loss [symbol] _{ineff}=ω·Ω(C(X;τ)) with ω:=ω(Y) depending on the ground truth Y.

The system 100 can further take into consideration which classes are included in the confidence sets. For example, the classification loss can be designed to penalize the “confusion” between pairs of classes. For example, if two diseases are frequently confused by doctors, it makes sense to train models that avoid confidence sets that contain both diseases. Reducing the probability of including both y and k classifications in the confidence set can be accomplished by using a positive entry L_{y,k}>0 in Eq. (12).

Further, the conformal training process of system 100 enables explicitly penalizing an “overlap” between groups of classes in the confidence set. For example, a medical professional may want to avoid concurrently including very high-risk conditions among low-risk ones in confidence sets, to avoid unwanted anxiety or tests for the patient. In order to reduce the probability of including a class from a particular group of classes K₁ in confidence sets that include any of classes K₀, the system can set L_{y,k}>0 for y∈K₀, k∈K₁ in Eq. (12).

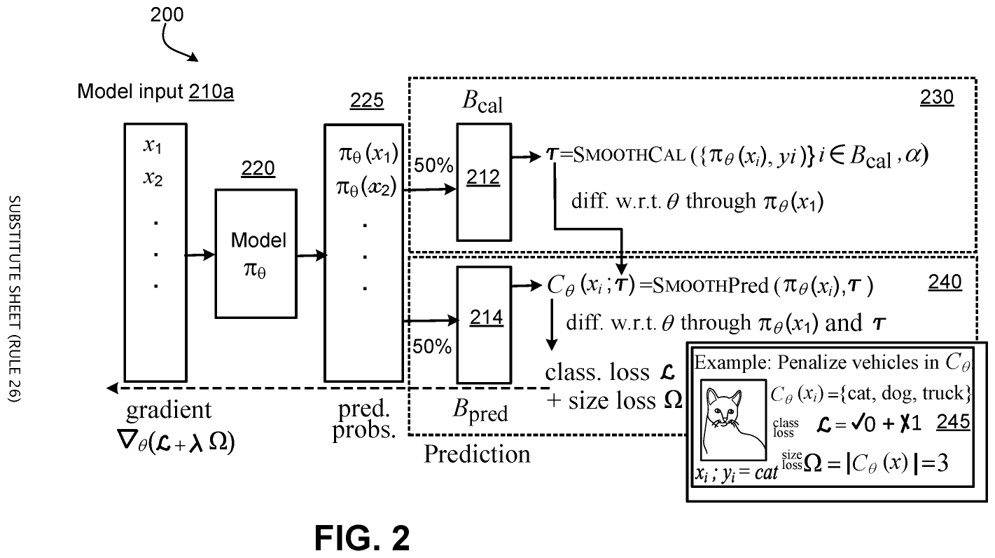

FIG. 2 shows an example of performing conformal training process 200 of a classification machine-learning model. For convenience, the conformal training process 200 will be described as being performed by a system of one or more computers located in one or more locations. For example, a conformal training system, e.g., the conformal training system 100 of FIG. 1, appropriately programmed in accordance with this specification, can perform the process 200.

As shown in FIG. 2, the classification model 220 is configured to process model inputs 210a of training examples to generate classification outputs 225. The system can split the training example dataset into a calibration batch 212 and a prediction batch 214, e.g., with a 50%-50% split.

The system applies a smooth calibration function 230 to the classification outputs generated from the calibration batch 212, the ground-truth labels of the calibration batch 212, and a user-specified significance level α to generate the threshold value τ. The threshold value τ is differentiable with respect to the model parameters of the classification model 220 through the classification outputs.

Then the system applies a smooth prediction function 240 on the classification outputs generated from the prediction batch 214, and the threshold value τ to generate the confidence scores C_{θ}(x;τ). The confidence scores C_{θ}(x;τ) are differentiable with respect to the model parameters of the classification model 220 through the classification outputs and the threshold value τ.

The system then compute a training loss based on the confidence scores C_{θ}(x;τ) predicted based on the prediction batch 214. The training loss can include a size loss (or inefficiency loss) Ω and a classification loss [symbol] . As shown in the example in 245, the classification loss [symbol] can be shaped to implement constraints on the confidence set, e.g., by penalizing a group of classes (e.g., vehicles) in confidence sets. Thus, the classification loss [symbol] can specify a positive number for the class “truck” when it is not the ground-truth label.

After the training loss is computed on the prediction batch, the system backpropagates the gradients of the training loss with respect to the model parameters to update the values of the model parameters.

FIG. 3 shows an example conformal classification system 300 for predicting a confidence set 345 for input data 310. The system 300 is an example of a system implemented as computer programs on one or more computers in one or more locations, in which the systems, components, and techniques described below can be implemented.

In general, the system 300 generates the confidence set 345 that includes one or more output classifications selected from a set of multiple classifications such that a probability of a true classification of the model input specified in the input data 310 being included in the confidence set is greater than or equal to a user-specified confidence level 316, e.g., 1−α. That is, the classification accuracy of the confidence set (in the sense that the confidence set includes the true classification) is guaranteed to satisfy the confidence level, which can be user-defined according to a specific application.

The system 300 includes a classification model 320 that has been conformally trained, e.g., by a conformal training system such as the system 100 of FIG. 1. That is, the classification model 320 has been trained end-to-end with a smooth calibration function (which determines a threshold value) and a smooth conformal prediction function. The parameters of the classification model 320 have been optimized to minimize a training loss (e.g., including an inefficiency loss) computed on a predicted confidence set based on a particular confidence level 1−α′ selected for the training.

The conformally trained classification model 320 receives the input data 310 as the model input, and processes the input data 310 to generate the classification output 325. The classification output 325 includes, for example, predicted probabilities for the model input being classified into each of a set of classifications. The system then generates the conformity scores 326 based on the classification output 325. The conformity scores 326 can be defined following any suitable conformal prediction formulation, such as the THR or APS formulations discussed with reference to FIG. 1.

In some implementations, the system 300 uses the same conformal prediction formulation (i.e., the same definition for the conformity scores) as have been used to conformally train the classification model 320. However, this is not mandatory requirement. After the classification model 320 has been conformally trained, any appropriate conformal prediction formulation can be used in the conformal classification system 300. For example, even if the THR formulation has been used to conformally train the classification model 320, the system 300 can still use another conformal prediction formulation, e.g., the APS formulation, to define the conformity scores 326 and predict the confidence set 345 based on a threshold value 335.

The threshold value 335 is determined by a calibration process using the calibration function 330 on a calibration training data set 312. The calibration process performed by the calibration function 330 is similar to the calibration process described with reference to the smooth calibration function 130 in FIG. 1. However, generally, the calibration function 330 is not a smooth function with regard to the input. Instead, the calibration function 330 is generally a non-differentiable function with respect to the input to the function, i.e., the classification output for the calibration set 312. For example, in the THR or APS formulations, the quantile operations will be conventional non-differentiable quantile operations instead of smoothed quantile operations for computing the threshold value 335 based on the confidence level 316 using the calibration function 330.

In some implementations, the confidence level 316 (1−α) for determining the calibrated threshold 335 matches the confidence level 1−α′ that was used to conformally train the classification model 320. However, this is not strictly required. In some implementations, the confidence level 316 (1−α) can be a different value from the confidence level 1−α′ that was used to conformally train the classification model 320. Thus, once the classification model 320 has been conformally trained, various conformal calibrations and predictions using different types of conformal prediction formulation and different confidence levels can be used with the trained classification model 320 to predict the confidence set 345.

After determining the threshold value 335, the system can use the threshold value to select classifications to be included in the predicted confidence set 345 based on the predicted probabilities for the classifications specified by the classification output 325. For example, the system can use Eq. 2 to determine the confidence set 345 if the THR formulation is used. In another example, the system can use Eq. 5 and 6 to determine the confidence set 345 if the APS formulation is used.

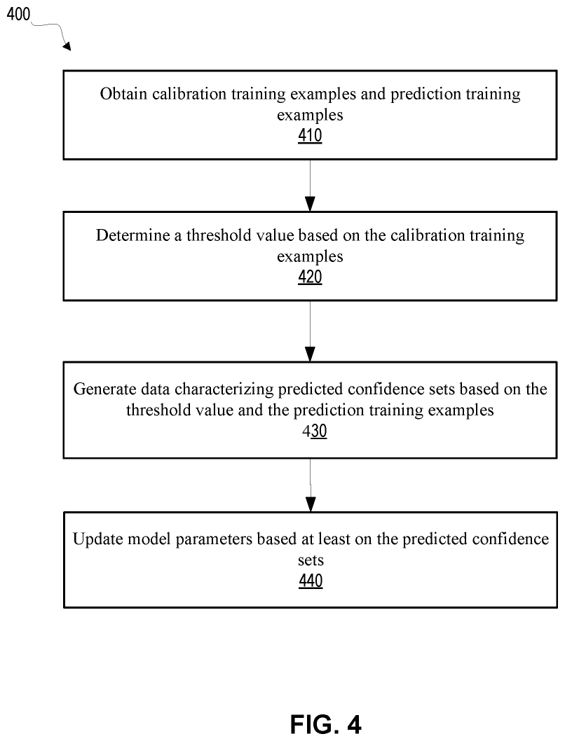

FIG. 4 is a flow diagram illustrating an example process 400 for performing conformal training of a classification model. For convenience, the process 400 will be described as being performed by a system of one or more computers located in one or more locations. For example, a conformal training system, e.g., the conformal training system 100 described with reference to FIG. 1, appropriately programmed in accordance with this specification, can perform the process 400.

In step 410, the system obtains calibration training examples and prediction training examples.

In some implementations, the system obtains a batch of training examples, and splits, e.g., via random sampling, the batch into the calibration training examples and the prediction training examples. Each training example includes a respective training model input and a respective classification label for the respective training model input.

In step 420, the system determines a threshold value based on the calibration training examples. In particular, for each calibration training example, the system processes the respective training model input of the respective calibration training example using the classification model, according to current values of the model parameters, to generate a respective classification output for the respective training model input. The system then determines a threshold value based at least on the classification outputs generated for the calibration training examples, the classification labels in the calibration training examples, and a confidence level.

In particular, to determine the threshold value, the system can process the classification outputs, the classification labels in the calibration training examples, and the confidence value with a smooth calibration function that outputs the threshold value. The smooth calibration function is differentiable with respect to the classification outputs, and thus is also differential with respect to the model parameters of the classification model.

For example, the system can compute, for each calibration training example, a respective conformity score using an appropriate formulation (e.g., the THR or APS formulations) based on the respective classification output and the respective classification label. The system then performs a smoothed implementation of a quantile operation on the conformity scores and the confidence level to determine the threshold value.

In step 430, the system generates data characterizing predicted confidence sets based on the threshold value and the prediction training examples.

In some implementations, for each prediction training example, the system processes the respective model input of the respective prediction training example using the classification model, according to the current values of the model parameters, to generate a respective classification output. The system then processes an input including the respective classification output and the threshold value with a smooth prediction function to generate a respective prediction output. In particular, the respective prediction output characterizes a predicted confidence set that includes one or more output classifications selected from the set of classifications such that a probability of a true classification of the respective model input being included in the predicted confidence set is greater than or equal to the confidence level.

The smooth prediction function is differentiable with respect to the input to the smooth prediction function, i.e., each of the classification output and the threshold value. Since each of the classification output and the threshold value is differentiable with respect to the model parameters of the classification model, the prediction output is also differentiable with respect to the model parameters of the classification model.

In one particular example, to generate the prediction output, the system computes the respective conformity scores for each classification output using the formulation chosen in the smooth calibration process, and uses the smooth prediction function to process the conformity scores and the threshold value to generate confidence scores that characterize the probabilities of each of the set of classifications being included in the confidence set.

In step 440, the system update model parameters of the classification model based at least on the predicted confidence.

In some implementations, the system computes a gradient with respect to the model parameters of a training loss that is computed based at least on the respective prediction outputs. In particular, the training loss includes an inefficiency loss that measures values characterizing the sizes of the predicted confidence sets characterized by the prediction outputs. For example, the value characterizing the size of the predicted confidence can be computed based on a sum of the confidence scores characterizing the probabilities of each classification being included in the confidence set.

In some implementations, the training loss further includes a classification loss that measures a classification error of the output classifications included in the predicted confidence set.

For example, the classification loss can include a first term that measures, for each respective prediction training example, an error of not including the true classification, as indicated by the respective classification label in the respective prediction training examples, in the predicted confidence set characterized by the prediction output.

The classification loss can further include a second term that measures, for each respective prediction training example, an error of including a specified classification that is not the true classification in the respective output confidence set. For example, the specified classification can be a classification contradictory to the true classification is determined, based on priori information, as a classification.

The system can shape the inefficiency loss and/or the classification loss to adapt to different application scenarios. For example, the system can implement the inefficiency loss as a weighted size that is computed based on the sizes of the respective predicted confidence sets scaled by weight coefficients for the set of classifications. For example, the system can assign a greater value for the weight coefficient to a particular classification that is less important to detect.

In another example, the system can include one or more additional terms in the classification loss to penalize two or more classifications being included together in the predicted confidence set. This can be useful for scenarios in which certain classifications may be contradictory to each other, or for scenarios in which certain classifications can easily be confused with each other.

After the gradients with respect to the model parameters of the training loss have been determined, the system can update the current values of the model parameters using the gradients. The system can update the parameters of the classification model (e.g., a classification neural network) using any appropriate backpropagation-based machine-learning technique, e.g., using the Adam or AdaGrad optimizers. In some implementations, the system can apply stochastic gradient descent on multiple batches of training examples, and repeat the above steps for each batch.

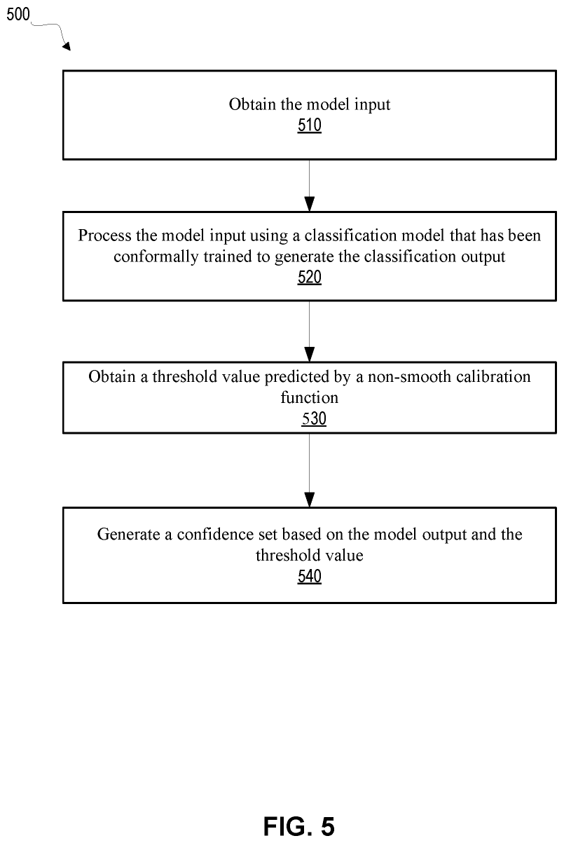

FIG. 5 is a flow diagram illustrating an example process for performing conformal classification. For convenience, the process 500 will be described as being performed by a system of one or more computers located in one or more locations. For example, a conformal classification system, e.g., the conformal classification system 300 described with reference to FIG. 3, appropriately programmed in accordance with this specification, can perform the process 500.

In step 510, the system obtains a model input. The model input can include any type of data to be analyzed, e.g., an image, a text, an audio signal, a sensor measurement, event log, and so on.

In step 520, the system processes the model input using a classification model that has been conformally trained to generate a classification output. The output data can specify classification information for the model input, e.g., one or more object categories of objects being depicted by an input image, one or more text categories of an input text, one or more status categories of a system, one or more health condition categories of a person, and so on. In particular, the classification output indicates, for each particular classification in a set of classifications, a predicted probability for the model input for the particular classification.

The classification model has been conformally trained, e.g., by a conformal training system such as the system 100 described with respect to FIG. 1. That is, the classification model has been trained end-to-end with a smooth calibration function (which determines a threshold value) and a smooth conformal prediction function. The parameters of the classification model have been optimized to minimize a training loss (e.g., including an inefficiency loss) computed on a predicted confidence set based on a particular confidence level 1−α′ selected for the training.

In step 530, the system obtains a threshold value predicted by a non-smooth calibration function according to a confidence level. The threshold value has been determined by a calibration process using the calibration function on a calibration training data set. The non-smooth calibration function is generally non-differentiable with respect to the input to the function, i.e., the classification output for the calibration set. For example, in the THR or APS formulations, the quantile operations will be conventional quantile operations instead of smoothed quantile operations for computing the threshold value based on the confidence level using the non-smooth calibration function.

In step 540, the system generates a confidence set based on the model output and the threshold value. In particular, the system can compare threshold value with the conformity scores computed based on the model output, and select classifications with corresponding conformity scores that satisfy a condition for the comparison result (e.g., according to Eq. 2 or Eq. 5 and 6), to be included in the output confidence set.

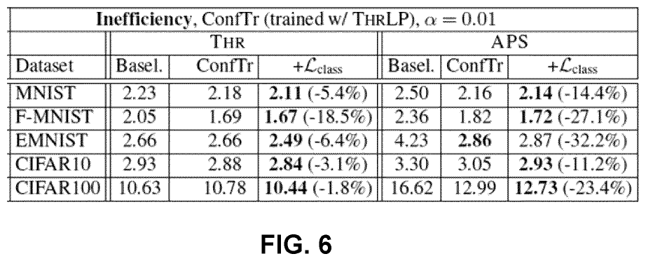

FIG. 6 shows a performance comparison between classification models trained using conformal training (ConfTr) and baseline classification models (Basel.) without conformal training in predicting the confidence set

The conformal training is based on the THRLP formulation with a significance level of α=0.01. After training, the trained classification models are used with a conformal prediction wrapper either using the THR formulation or the APS formulation to predict confidence sets.

FIG. 6 summarizes the inefficiencies, i.e., a measure indicating the sizes of the predicted confidence sets for various methods based on 5 test datasets. As shown by the data in FIG. 6, conformal training (ConfTr) significantly improves inefficiency in the predicted confidence set compared to baseline. Further, it is shown that including the classification loss (+ [symbol] _{class}) generally further improves on the inefficiency.

Further, FIG. 6 shows that, even if the conformal predictor formulation used on the conformal prediction wrapper does not match the conformal predictor formulation used in conformal training (e.g., THR or APS vs. THRL), conformal training still provides significant improvement on inefficiency. Thus, once a classification model has been conformally trained, it can be used with different conformal prediction wrappers depending on the application to effectively predict the confidence set.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions. Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine: in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices: magnetic disks, e.g., internal hard disks or removable disks: magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well: for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user: for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine-learning models can also include, for example, special-purpose hardware accelerator units for processing common and compute-intensive parts of machine-learning training or production, i.e., inference, workloads.

Machine-learning models can be implemented and deployed using a machine-learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method for training a conformal classification machine-learning model, wherein the conformal classification machine learning model comprises:
a classification model having a plurality of classification model parameters, the classification model being configured to process a model input to generate a classification output that indicates, for each particular classification in a set of classifications, a predicted probability for the model input for the particular classification, and
a prediction function configured to process an input including the classification output to generate data identifying, for the model input, a confidence set that includes one or more output classifications selected from the set of classifications such that a probability of a true classification of the model input being included in the confidence set is greater than or equal to a confidence level, the method comprising:
obtaining a set of calibration training examples, each calibration training example including a respective training model input and a respective classification label for the respective training model input;
for each respective calibration training example in the set of calibration training examples, processing the respective training model input of the respective calibration training example using the classification model, according to current values of the classification model parameters, to generate a respective classification output for the respective training model input;
determining a threshold value based at least on the classification outputs generated for the calibration training examples, the classification labels in the calibration training examples, and the confidence level;
obtaining a set of prediction training examples, each prediction training example including a respective training model input and a respective classification label for the respective training model input;
for each respective prediction training example in the set of prediction training examples, performing:
processing the respective model input of the respective prediction training example using the classification model, according to the current values of the classification model parameters, to generate a respective classification output; and
processing an input including the respective classification output and the threshold value with the prediction function to generate a respective prediction output, wherein the prediction function is differentiable with respect to the respective classification output; and
determining a gradient with respect to the classification model parameters of a training loss that includes a inefficiency loss that at least measures, for each respective prediction training example, a value indicating a size of the respective output confidence set in the respective prediction output generated for the respective prediction training example; and
updating the current values of the parameters of the classification model parameters using the gradient.

2. The method of claim 1, wherein the respective prediction output includes a confidence score for each classification in the set of classifications, wherein the respective confidence score characterizes a respective probability of the respective classification being included in the predicted confidence set.

3. The method of claim 1, wherein the classification model includes a neural network.

4. The method of claim 1, wherein the training loss further includes a classification loss that measures a classification error of the output classifications included in the predicted confidence set characterized by the respective prediction output.

5. The method of claim 4, wherein the classification loss includes a first term that measures, for each respective prediction training example, an error of not including the true classification in the predicted confidence set characterized by the prediction output.

6. The method of claim 4, wherein the classification loss includes a second term that measures, for each respective prediction training example, an error of including a specified classification that is not the true classification in the respective output confidence set.

7. The method of claim 6, wherein the specified classification is determined, based on priori information, as a classification contradictory to the true classification.

8. The method of claim 1, wherein the prediction function includes a smoothed implementation of a threshold function applied to a difference between a first value computed based on the classification output and a second value computed based on the threshold value.

9. The method of claim 8, wherein for each respective prediction training example in the set of prediction training examples, the first value is computed as the predicted probability indicated by the respective classification output for a particular classification.

10. The method of claim 8, wherein for each respective prediction training example in the set of prediction training examples, the first value is computed as a sum of a plurality of predicted probabilities indicated by the respective classification output for a plurality of classifications from the set of classifications.

11. The method of claim 8, wherein the smoothed threshold function is a sigmoid function.

12. The method of claim 1, wherein determining the threshold value comprises:
processing the classification outputs from the classification model, the classification labels in the calibration training examples, and the confidence value with a calibration function that outputs the threshold value, wherein the calibration function is differentiable with respect to the classification outputs.

13. The method of claim 12, wherein the calibration function is configured to:
compute, for each respective calibration training example in the set of calibration training examples, a respective conformity score based on the respective classification output and the respective classification label; and
perform a smoothed implementation of a quantile operation on the conformity scores and the confidence level.

14. The method of claim 1, further comprising:
obtaining a batch of training examples; and
randomly sampling the batch of training examples to generate the set of calibration training examples and the set of prediction training examples.

15. The method of claim 14, wherein the batch of training examples is a first batch of training examples, and the method further comprises:
obtaining additional batches of training examples; and
repeatedly performing training of the classification machine-learning model on each of the additional batches of training examples.

16. The method of claim 1, wherein: the inefficiency loss measures a weighted size that is computed based on the sizes of the respective predicted confidence sets scaled by weight coefficients for the set of classifications.

17. The method of claim 1, wherein:
the model input comprises sensor data, and the classifications correspond to object categories;
the model input comprises medical data, such as physiological measurements, of a patient and the classifications each correspond to a different diagnosis for the patient; and/or
the model input comprise observations of the environment and the classifications each correspond to a different state of the environment.

18. A computer-implemented method for performing conformal classification, comprising:
obtaining the classification model that has been trained using the method of claim 1;
obtaining a model input;
processing the model input with the classification model to generate a classification output that indicates, for each particular classification in a set of classifications, a predicted probability for the model input for the particular classification;
obtaining a threshold value that has been predicted by a non-smooth calibration function based on a calibration data set and according to a confidence value; and
processing the model output and the threshold value to generate a confidence set that includes one or more output classifications selected from the set of classifications.

19. The method of claim 18, further comprising performing, based at least in part on the confidence set, one or more of:
controlling a robot or a vehicle, wherein the model input comprises sensor data and the classifications correspond to object categories;
providing one or more medical diagnoses, wherein the model input comprises medical data, such as physiological measurements, of a patient and the classifications each correspond to a different diagnosis for the patient; and
controlling an agent to perform a task in an environment, wherein the model input comprise observations of the environment and the classifications each correspond to a different state of the environment.

20. A system comprising:
one or more computers; and
one or more storage devices storing instructions that, when executed by the one or more computers, cause the one or more computers to perform operations for training a conformal classification machine-learning model, wherein the conformal classification machine learning model comprises:
a classification model having a plurality of classification model parameters, the classification model being configured to process a model input to generate a classification output that indicates, for each particular classification in a set of classifications, a predicted probability for the model input for the particular classification, and
a prediction function configured to process an input including the classification output to generate data identifying, for the model input, a confidence set that includes one or more output classifications selected from the set of classifications such that a probability of a true classification of the model input being included in the confidence set is greater than or equal to a confidence level, the operations comprising:
obtaining a set of calibration training examples, each calibration training example including a respective training model input and a respective classification label for the respective training model input;
for each respective calibration training example in the set of calibration training examples, processing the respective training model input of the respective calibration training example using the classification model, according to current values of the classification model parameters, to generate a respective classification output for the respective training model input;
determining a threshold value based at least on the classification outputs generated for the calibration training examples, the classification labels in the calibration training examples, and the confidence level;
obtaining a set of prediction training examples, each prediction training example including a respective training model input and a respective classification label for the respective training model input;
for each respective prediction training example in the set of prediction training examples, performing:
processing the respective model input of the respective prediction training example using the classification model, according to the current values of the classification model parameters, to generate a respective classification output; and
processing an input including the respective classification output and the threshold value with the prediction function to generate a respective prediction output, wherein the prediction function is differentiable with respect to the respective classification output; and
determining a gradient with respect to the classification model parameters of a training loss that includes a inefficiency loss that at least measures, for each respective prediction training example, a value indicating a size of the respective output confidence set in the respective prediction output generated for the respective prediction training example; and
updating the current values of the parameters of the classification model parameters using the gradient.

21. One or more computer-readable storage media storing instructions that when executed by one or more computers cause the one or more computers to perform operations for training a conformal classification machine-learning model, wherein the conformal classification machine learning model comprises:
a classification model having a plurality of classification model parameters, the classification model being configured to process a model input to generate a classification output that indicates, for each particular classification in a set of classifications, a predicted probability for the model input for the particular classification, and
a prediction function configured to process an input including the classification output to generate data identifying, for the model input, a confidence set that includes one or more output classifications selected from the set of classifications such that a probability of a true classification of the model input being included in the confidence set is greater than or equal to a confidence level, the operations comprising:
obtaining a set of calibration training examples, each calibration training example including a respective training model input and a respective classification label for the respective training model input;
for each respective calibration training example in the set of calibration training examples, processing the respective training model input of the respective calibration training example using the classification model, according to current values of the classification model parameters, to generate a respective classification output for the respective training model input;
determining a threshold value based at least on the classification outputs generated for the calibration training examples, the classification labels in the calibration training examples, and the confidence level;
obtaining a set of prediction training examples, each prediction training example including a respective training model input and a respective classification label for the respective training model input;
for each respective prediction training example in the set of prediction training examples, performing:
processing the respective model input of the respective prediction training example using the classification model, according to the current values of the classification model parameters, to generate a respective classification output; and
processing an input including the respective classification output and the threshold value with the prediction function to generate a respective prediction output, wherein the prediction function is differentiable with respect to the respective classification output; and
determining a gradient with respect to the classification model parameters of a training loss that includes a inefficiency loss that at least measures, for each respective prediction training example, a value indicating a size of the respective output confidence set in the respective prediction output generated for the respective prediction training example; and
updating the current values of the parameters of the classification model parameters using the gradient.
